(19) United States
(12) Patent Application Publication
(10) Pub. No.: US 2012/0096745 A1
(43) Pub. Date: Apr. 26, 2012

(54) SHOE INSOLE FOR METATARSAL RELIEF

(76) Inventor: ANDREW DONATO, Toronto (CA)

(21) Appl. No.: 12/910,168

(22) Filed: Oct. 22, 2010

(51) Int. Cl. A61F 5/14; A43B 7/14

(52) U.S. Cl. 36/174

(57) ABSTRACT

A shoe or footwear with an insole or base configured for relieving pressure on the metatarsal region of the foot and help relieve the effects of metatarsalgia. The insole comprises a cavity or cut out in the area of the metatarsal region of the foot and is configured to receive at least a portion of the metatarsal region and alleviate or eliminate direct pressure from the insole against the metatarsal region.

### FIELD OF THE INVENTION

This invention relates to shoes, and more particularly to an insole and a shoe design for relieving pressure on the metatarsal region of the foot.

### BACKGROUND OF THE INVENTION

Metatarsalgia is a common foot ailment suffered by many. Metatarsalgia is a condition where the muscle or tissue in the metatarsal region of the foot begins to break down and the condition can make walking, running or even standing very uncomfortable and painful. To compensate for the pain and discomfort, a person may alter their gait or posture which can lead to further complications, other joint ailments and maladies.

Known methods of relieving the pain associated with metatarsalgia typically comprise using padding in the forefoot area of the shoe to cushion the ball of foot. Such approaches attempt to replace the internal tissue loss with external padding. While they may be effective for certain people, the pressure of the padding against the ball of the foot can still cause discomfort and pain to sufferers of metatarsalgia.

Accordingly, there remains a need for improvements in the art.

### BRIEF SUMMARY OF THE INVENTION

The present invention is directed to an insole insert, a shoe and/or an insole/sole configuration for a shoe or footwear that relieves pressure from the metatarsal region or other areas of the foot.

According to one embodiment, the present invention comprises an insole for a shoe configured for relieving pressure from a metatarsal region of a foot, the insole comprising a base configured for fitting inside the shoe, a cut out formed in said base, the cut out forming a void area underneath the metatarsal region of the foot, wherein the void area allows the metatarsal region to avoid substantial contact with the insole of the shoe.

According to another embodiment, the present invention comprises a shoe suitable for relieving symptoms associated with metatarsalgia arising from pressure on the metatarsal region of a wearer's foot, the shoe comprising, a shoe upper, a sole and an insole, the shoe upper being joined to the sole, and the insole being configured to fit inside the shoe upper and rest on the sole, and the insole includes an open cavity formed in the area corresponding to the metatarsal region of the foot, wherein said open cavity provides a non-contactive region for the metatarsal region of the foot so as to relieve pressure or contact against the metatarsal region.

According to another embodiment, the present invention comprises an insole for a shoe configured for relieving pressure from a metatarsal region of a foot, the insole comprising a base configured for fitting inside the shoe, a cavity formed in the base, the cavity forming a void area underneath the metatarsal region of the foot and having a peripheral edge, the peripheral edge providing support to region of the foot adjacent to the metatarsal region of the foot so as to relieve direct pressure against the metatarsal region of the foot.

Other aspects and features of the present invention will become apparent to those ordinarily skilled in the art upon review of the following exemplary embodiments of the invention in conjunction with the accompanying figures.

### BRIEF DESCRIPTION OF THE DRAWINGS

Reference will now be made to the accompanying drawings, which show by way of example, embodiments according to the present invention, and in which:

FIG. 1 shows a shoe with an insole/sole configuration for relieving pressure against the metatarsal region of a foot according to an embodiment of the present invention;

FIG. 2 is a cut-away or section view of the shoe of FIG. 1 taken along the line A-A and showing a foot inserted inside the shoe;

FIG. 3 is a top view of an insole with a metatarsal cavity or cut-out according to an embodiment of the present invention;

FIG. 4 is a perspective side view of a sole assembly with a metatarsal cavity according to an embodiment of the present invention;

FIG. 5(a) is front sectional view of the sole assembly of FIG. 4 taken along line B-B;

FIG. 5(b) is back sectional view of the sole assembly of FIG. 4 taken along line B-B;

FIG. 6(a) is a left side sectional view of the sole assembly of FIG. 4 taken along line C-C; and

FIG. 6(b) is a right side sectional view of the sole assembly of FIG. 4 taken along line C-C.

Like reference numerals indicate like elements or components in the drawings.

### DETAILED DESCRIPTION OF THE EMBODIMENTS

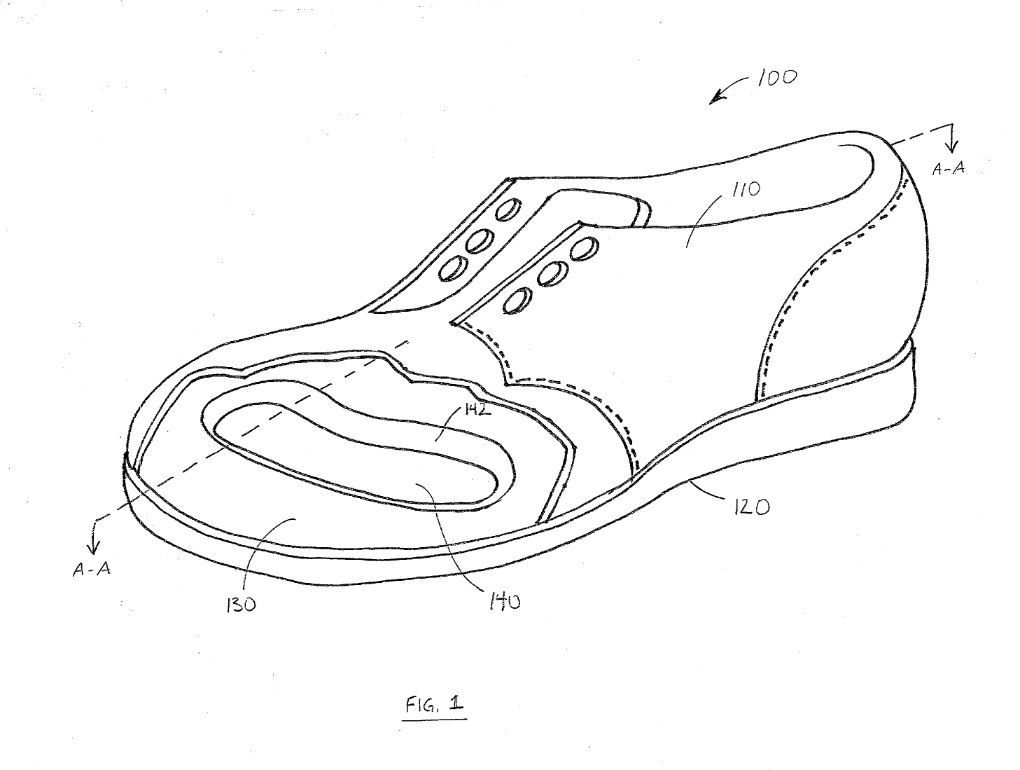

Reference is first made to FIG. 1, which shows a piece of footwear or shoe configured with a metatarsal pressure cavity or cut out according to an embodiment of the present invention. The shoe is indicated generally by reference 100 and comprises a shoe upper 110, a shoe or under sole 120 and an insole 130. As will be described in more detail below, the insole 130 can be formed integrally with the sole 120 or comprise a separate piece that is inserted into the shoe 100 and can be permanently affixed or press fit for removal or replacement. It will also be appreciated that while embodiments of the present invention are described in the context of a piece of footwear such as a shoe, the embodiments find wider applicable to other types of shoes and specialty footwear, such as sandals, hiking boots, ski boots, ice skates, inline skates, skin diving fins, etc.

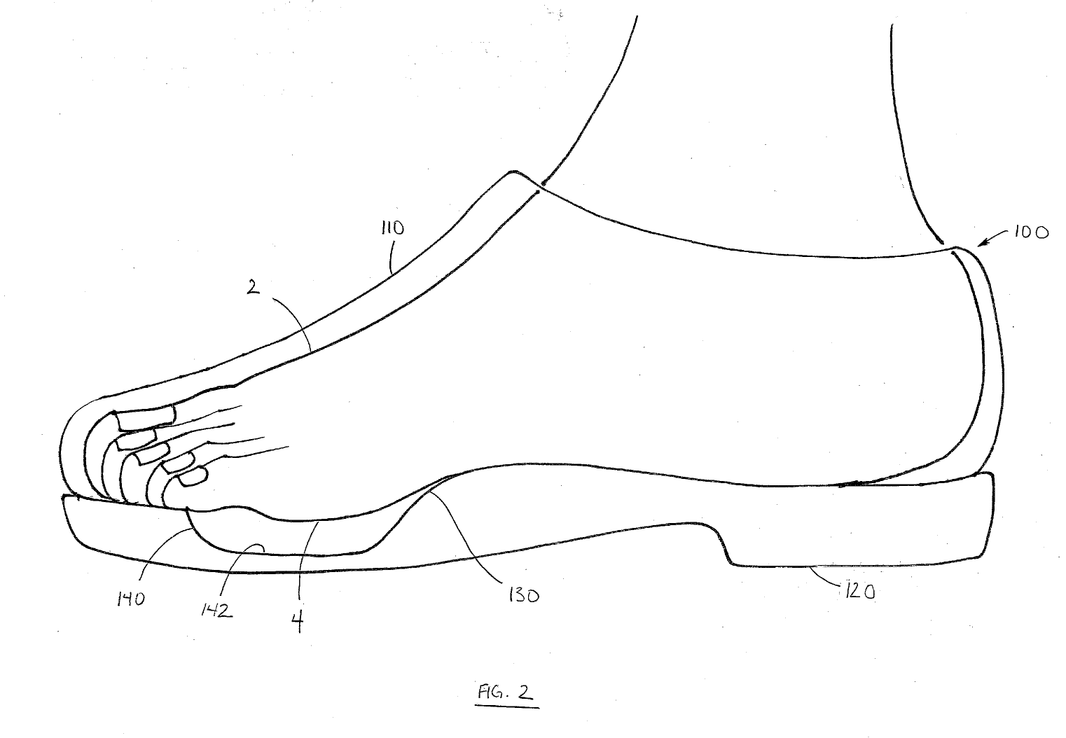

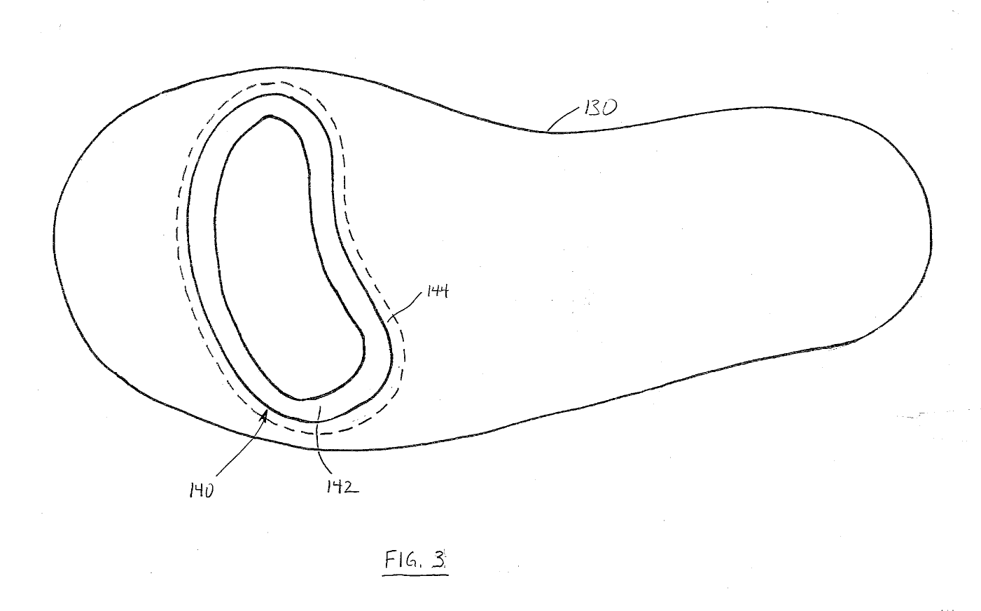

As shown in FIG. 1, the insole 130 includes a cut out or a cavity section indicated generally by reference 140. The cavity 140 is located in a section of the insole 130 that substantially corresponds to the metatarsal region of the foot, which is typically around the ball of the foot (for example, FIG. 2 shows a foot indicated by reference 2 with a metatarsal region indicated by reference 4). According to an embodiment, the cavity or cut out 140 is formed or cut through the insole 130. According to another aspect, the cut out 140 comprises a side edge 142 extending around the cut out. As shown in FIG. 2 and also FIG. 3, the side edge 142 can be formed with relief, e.g. on an angle or with a rounded profile. According to another aspect, the side edge 142 can comprise a substantially vertical cut or edge. As shown in FIG. 2, the cavity 140 is open at the top, i.e. at the surface of the insole 130, and as such provide an open void or recess into which the metatarsal region 4 of the foot 2 can extend without any substantial contact with the insole 130. This has the effect of alleviating or lessening pain in the metatarsal region 4 by eliminating or substantially reducing direct contact with the insole 130 and the metatarsal region 4. It has been found that by providing a void or cavity below the metatarsal region 4 no other padding or cushioning is required in the area of the metatarsal region 4 and the lack of any direct contact or pressure on the metatarsal region 4 provides comfort to the wearer of the shoe 100. It will be appreciated that the size of the cavity or cut out 140, e.g. diameter for a generally round or oval shaped cut-out, or width and length for a generally rectangular or oblong shaped cut-out, will depend on the size of the foot, the size of metatarsal region or the size of the effected metatarsal region, and/or the size of the shoe or the insole. In most cases, the cut out 140 will be sized to avoid substantial contact or direct contact with the metatarsal region or an area of the metatarsal region which may be sensitive to pressure or affected by pressure or contact. As such, the size and/or shape of the cut-out 140 can be customized or varied based on such factors or parameters.

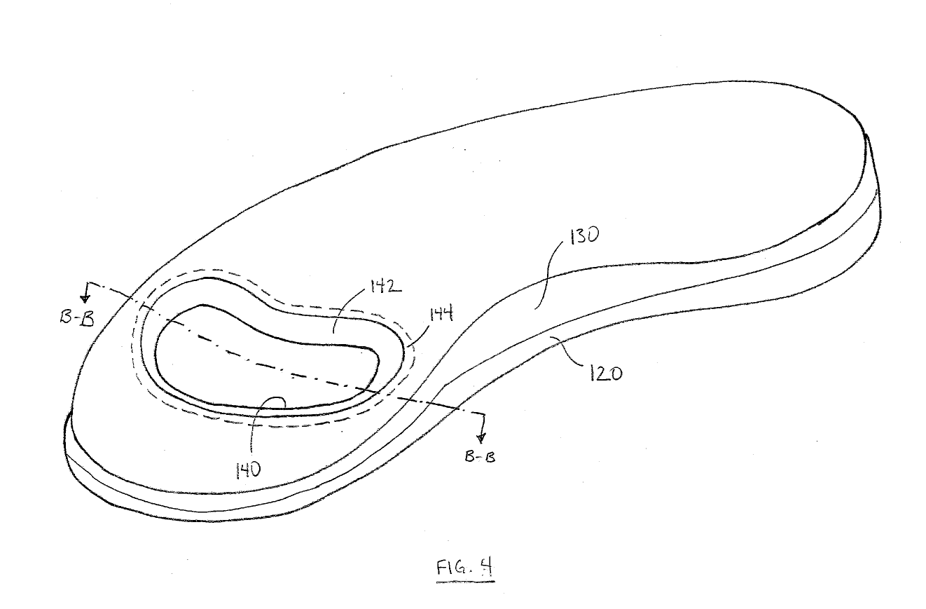

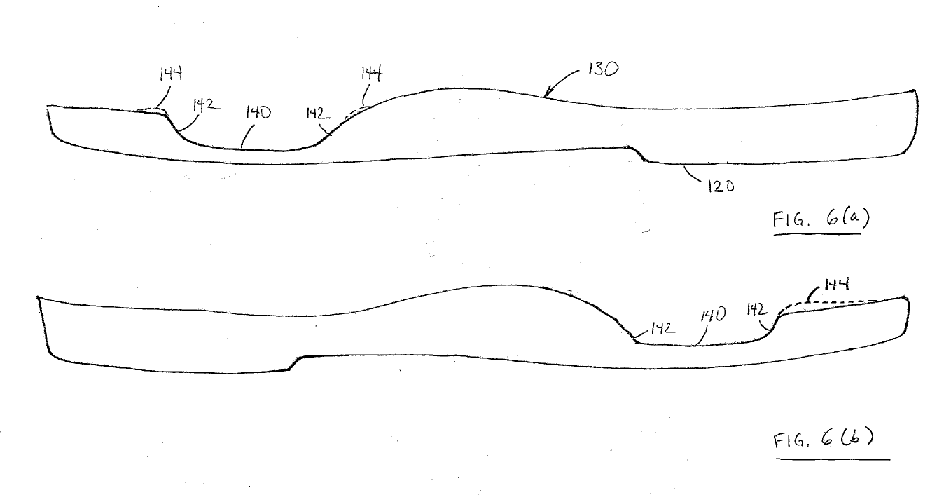

According to another embodiment, the insole 130 can include a support surface or support structure as shown in FIG. 4 and FIGS. 6(a) and 6(b), and indicated generally by reference 144. The support surface 144 extends substantially around the perimeter of the cut out 140. According to an embodiment, the support surface 144 is configured to reinforce the perimeter around the cut out along the top surface of the insole 130. According to another embodiment, the support surface 144 is configured to provide a cushioning strip or area for contacting the perimeter of the metatarsal region 4 (FIG. 2) of the foot 2 (FIG. 2). According to an embodiment, the support surface 144 slopes away from the cut out 140, for example, so as not to provide a pressure point. According to another aspect, the support surface 144 is formed from a material that has a cushioning characteristic, for example, a gel strip, that is co-molded with the insole or otherwise attached or affixed to the periphery of the cavity 140.

According to another embodiment, the insole 130 comprises a separate component or insert as depicted in FIG. 3. According to this embodiment, the insole 130 is inserted into the shoe 100 during assembly or manufacturing, or comprises an after-market insole that is used to replace the existing insole, with the metatarsal relief configured insole 130 as described herein. According to another embodiment as shown in FIG. 4, the insole 130 is formed as integral component of the sole 120. According to another embodiment, the insole 130 is glued or otherwise affixed to the sole 120. According to an embodiment, the cavity 140 can be configured to extend through the insole 130 and the sole 120 in order to achieve the required depth or clearance for the foot shape of the wearer, and the dimensions/depth of the cavity 140 will depend on the thickness of the insole 130 and/or the thickness of the sole 120. It will be appreciated that the insole 130 and the sole 120 can be configured according to known shoe manufacturing techniques as will be understood by one skilled in the shoe making art.

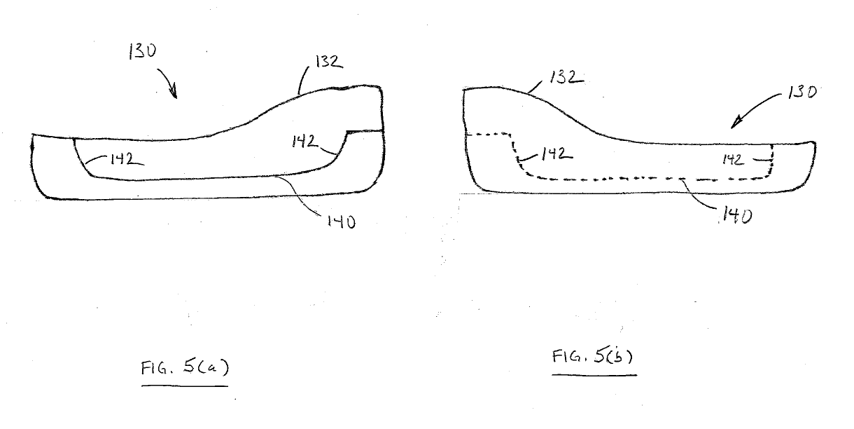

Reference is next made to FIGS. 5(a) and 5(b), which show respective front and back sectional views of the insole 130 and sole 120 arrangement according to an embodiment of the present invention. The sectional views are taken through the line B-B in FIG. 4. As shown, the cut out or cavity 140 extends substantially across the width of the insole 130 or sole 120. As also shown, the insole 130 includes an arch support or instep section indicated generally by reference 132.

In summary and according to an embodiment, the shoe with metatarsal relief comprises an insole with a cavity or cut out in an area corresponding substantially to the metatarsal region of the foot. The cavity is open and provides an open-air void for receiving at least a portion of the metatarsal region and thereby alleviates or prevents pressure or contact either direct or indirect from the insole or sole pressing against the metatarsal region of the foot.

The present invention may be embodied in other specific forms without departing from its spirit or essential characteristics. The embodiments described and disclosed are to be considered in all aspects only as illustrative and not restrictive. The scope of the invention is, therefore, indicated by the appended claims rather that by the foregoing description. All changes which come within the meaning and range of equivalency of the claims are to be embraced within their scope.

What is claimed is:

1. An insole for a shoe configured for relieving pressure from a metatarsal region of a foot, said insole comprising:
a base configured for fitting inside the shoe;
a cavity formed in said base, said cavity forming a void area underneath the metatarsal region of the foot; and
wherein the void area allows the sole of the foot to rest on the base without excess pressure being asserted on the metatarsal region of the foot.

2. The insole as claimed in claim 1, wherein said cavity comprises a perimeter area surrounding at least a portion of the void area, and said perimeter area being configured to provide a support surface for supporting at least a portion of the foot surrounding or adjacent the metatarsal region.

3. The insole as claimed in claim 1, wherein said cavity comprises a cut out extending through the base of said insole.

4. The insole as claimed in claim 3, wherein said cavity comprises a perimeter area surrounding at least a portion of the void area, and said perimeter area being configured to reinforce at least a portion of the edge of the cavity.

5. The insole as claimed in claim 2, wherein said insole comprises a thickness in the range of 0.125 inches to 0.500 inches, and the cut out extends completely through the insole.

6. A shoe suitable for relieving symptoms associated with metataralgia arising from pressure on the metatarsal region of a wearer's foot, said shoe comprising:
a shoe upper;
a sole;
an insole;
said shoe upper being joined to said sole, and said insole being configured to fit inside said shoe upper and rest on said sole; and
said insole includes an open cavity formed in the area corresponding to the metatarsal region of the foot, wherein said open cavity provides a non-contact region for receiving the metatarsal region of the foot to relieve pressure against the metatarsal region.

7. The shoe as claimed in claim 5, wherein said insole is formed integrally with said sole, and said cavity extending through said insole and having a bottom portion defined by said sole.

8. The shoe as claimed in claim 7, wherein said cavity comprises a perimeter area surrounding at least a portion of the void area, and said perimeter area being configured to reinforce at least a portion of the edge of the cavity.

9. The shoe as claimed in claim 6, wherein said cavity has a depth in the range 0.125 to 0.500 inches.

10. The shoe as claimed in claim 5, wherein comprises a perimeter area surrounding at least a portion of said cavity, and said perimeter area being configured to provide a support surface for supporting at least a portion of the foot surrounding or adjacent the metatarsal region.

11. An insole for a shoe configured for relieving pressure from a metatarsal region of a foot, said insole comprising:
a base configured for fitting inside the shoe;
a cavity formed in said base, said cavity forming an open area underneath the metatarsal region of the foot;
said cavity having a peripheral edge, said peripheral edge providing support to region of the foot adjacent to the metatarsal region of the foot so as to relieve direct pressure against the metatarsal region of the foot.

12. The insole as claimed in claim 11, wherein said base has a thickness in the range of 0.125 to 0.500 inches.

13. The insole as claimed in claim 11, wherein cavity comprises a cut out section and extends through the insole.

14. The insole as claimed in claim 13, wherein said base has a thickness in the range of 0.125 to 0.500 inches.

15. The insole as claimed in claim 11, wherein said peripheral edge comprises a material having a cushioning characteristic.
